(19) United States
(12) Patent Application Publication
(10) Pub. No.: US 2008/0248104 A1
(43) Pub. Date: Oct. 9, 2008

(54) COMPOSITION AND METHOD FOR REDUCING INFLAMMATION

(75) Inventors: Ken Clement, Mississauga (CA); Shan Chaudhuri, Mississauga (CA); Marvin A. Heuer, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: IOMEDIX DEVELOPMENT INTERNATIONAL SRL, St. Michael (BB)

(21) Appl. No.: 11/867,491

(22) Filed: Oct. 4, 2007

Related U.S. Application Data
(60) Provisional application No. 60/863,171, filed on Oct. 27, 2006.

(51) Int. Cl. A61K 9/48; A61K 38/48

(52) U.S. Cl. 424/456; 424/94.65

(57) ABSTRACT

The present invention relates to a composition and method for the simultaneous alleviation of the symptoms of inflammation brought about by innate immune responses wherein said composition acts by inhibiting leukotrienes, chemotactic cytokines and cyclooxygenases. The composition of the present invention comprises at least an extract of Butterbur, Bromelain and an extract of White Willow bark.

### RELATED APPLICATIONS

The application is related to and claims benefit of priority to U.S. Provisional Patent Application Ser. No. 60/863,171 entitled “Composition and Method for Reducing Inflammation,” filed Oct. 27, 2006, the disclosure of which is hereby fully incorporated by reference.

### FIELD OF THE INVENTION

The present invention relates to a composition and method for reducing inflammation. Specifically, the present invention relates to a composition and method for reducing inflammation brought about by innate immune responses in a mammal.

### BACKGROUND OF THE INVENTION

Inflammation is a complex defense response by the immune system, usually to infection or tissue and/or cellular damage. The innate immune response is the non-specific response mounted by a mammalian immune system and is comprised of cells such as leukocytes (natural killer cells, mast cells, eosinophils, basophils) and phagocytes (neutrophils, macrophages, dendritic cells) and the molecules they secrete such as histamine and various cytokines; and the complement system of circulating proteins which assist several aspects of the immune system in response to infection, allergens or tissue and/or cellular damage. There are several common clinical symptoms associated with inflammation including pain, localized redness, swelling and heat at the site of inflammation.

Leukotrienes are secreted lipid mediators of the inflammation response produced by leukocytes from arachidonic acid, which is a precursor for several mediators involved in inflammation. Production of leukotrienes is increased during inflammation. Moreover, leukotrienes can produce and sustain the symptoms of inflammation (Hedqvist P, Gautam N, Lindbom L. Interactions between leukotrienes and other inflammatory mediators/modulators in the microvasculature. Am J Respir Crit Care Med. 2000 February; 161(2 Pt 2):S117-9). Therapies targeting leukotrienes have proven efficacious for treating conditions involving inflammation such as asthma and allergies (Salvi S S, Krishna M T, Sampson A P, Holgate S T. The anti-inflammatory effects of leukotriene-modifying drugs and their use in asthma. Chest. 2001 May; 119(5):1533-46; Peters-Golden M, Gleason M M, Togias A. Cysteinyl leukotrienes: multi-functional mediators in allergic rhinitis. Clin Exp Allergy. 2006 June; 36(6):689-703).

The cyclooxygenase family of enzymes is involved in the formation of mediators of inflammation by converting arachidonic acid to various prostanoids, which mediate different aspects of the inflammatory response. Inhibition of cyclooxygenase activity is known to reduce pain and inflammation. Some of the most common and widely-used drugs such as aspirin/acetylsalicylic acid, ibuprofen and acetaminophen are cyclooxygenase inhibitors (Chandrasekharan N V, Dai H, Roos K L, Evanson N K, Tomsik J, Elton T S, Simmons D L. COX-3, a cyclooxygenase-1 variant inhibited by acetaminophen and other analgesic/antipyretic drugs: cloning, structure, and expression. Proc NatI Acad Sci USA. 2002 Oct. 15; 99(21):13926-31).

Chemotactic cytokines, or chemokines, are small molecules that specifically activate target cells and promote their migration. In inflammation, chemokines at the site of challenge attract immune cells from the blood toward areas of inflammation to facilitate their function. Anti-inflammatory drugs such as corticosteroids act by inhibiting the expression of various chemokines.

Inappropriate, excessive or chronic immune response can cause disease (Kulmatycki K M, Jamali F. Drug disease interactions: role of inflammatory mediators in disease and variability in drug response. J Pharm Pharm Sci. 2005 Dec. 16; 8(3):602-25). Allergies are hypersensitivities which result in inflammatory immune responses which may be local, as in the case with common allergies, or systemic which causes anaphylaxis. Examples of sites that are often afflicted with local allergies include the nose (allergic rhinitis), the eyes (allergic conjunctivitis) and airways (asthma).

Both common, currently used steroidal anti-inflammatory drugs (SAIDs) and non-steroidal anti-inflammatory drugs (NSAIDs) have been successful for treating acute inflammation but have not been successful at treating more chronic inflammatory conditions such as rheumatoid arthritis. Active constituents from plant sources such as flavonoids have demonstrated anti-inflammatory properties (Kim H P, Son K H, Chang H W, Kang S S. Anti-inflammatory plant flavonoids and cellular action mechanisms. J Pharmacol Sci. 2004 November; 96(3):229-45). These active plant constituents may act by targeting a number of different mechanisms or mediators of inflammation such as inhibiting histamine production by mast cells and inhibiting the activities of enzymes such as cyclooxygenase and phospholipase A2.

By modulating several different aspects of the innate immune response with safe, natural and well-tolerated substances in an effective amount it is possible to provide therapeutic relief from inflammation and associated symptoms.

### SUMMARY OF THE INVENTION

The present invention according to one embodiment thereof, provides for a nutritional composition directed towards reducing inflammation brought about by innate immune responses comprising an extract of Butterbur, Bromelain and an extract of White Willow bark. A method for the same is also provided by administration of said nutritional composition to a human in need thereof.

### DETAILED DESCRIPTION OF THE INVENTION

In the following description, for the purposes of explanations, numerous specific details are set forth in order to provide a thorough understanding of the present invention. It will be apparent, however, to one skilled in the art that the present invention may be practiced without these specific details.

As used herein, “an effective amount” refers to an amount of a given substance or composition effective to provide alleviation of symptoms inflammation brought about by innate immune responses when administered acutely or over a period of time in accordance with a predetermined dosage regimen.

A used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

As used herein the term ‘unmodified-release’ format is understood to be defined as pertaining to the dissolution and bioavailability profile of an ingested dietary ingredient wherein no additional modifications, be it chemical or physical, have been made to the ingredient with the specific intent to alter the dissolution or bioavailability profile from that of ingredient in a naturally occurring form. It is also understood that unmodified-release is, essentially, immediate-release of active ingredients. This is further understood to be traditional- or conventional-release format where no slow-, delayed- or extended-release effect is incorporated.

As used herein the term ‘controlled-release’ format is understood to be defined as a formulation of active ingredients and appropriate excipients in a specific format to facilitate a controlled- or non-immediate-release of active ingredients. The components of a controlled-release format may have been subjected to additional modifications, be it chemical or physical, with the specific intent to alter the dissolution or bioavailability profile from that of ingredient in a naturally occurring form.

As used herein the term ‘slow-release’ format is understood to be defined as a controlled-release format wherein the release of active ingredients are delayed for a period of time or gradually released over an extended period of time. This is accomplished through the use of specific excipients and may include structural features designed to facilitate controlled-release. It is further understood that a slow-release format releases active ingredients at a rate slower than immediate-release.

As used herein the term ‘delayed-release’ format is understood to be defined essentially as a controlled-release format wherein the components of the delayed-release format have undergone specific modifications, be it physical or chemical, to facilitate the release of active ingredients at a specific time after ingestion. It is further understood that delayed-release formats, release active ingredients at a period of time later than unmodified release.

As used herein the term ‘quick-release’ format is understood to be defined essentially as ‘unmodified-release’, as defined above. However, the term ‘quick-release’ may further include components having modifications, chemical or physical, to enhance the rate of dissolution or bioavailability of active ingredients.

The composition presented herein and those to be used in conjunction with methods presented herein are considered to be nutritional compositions useful for reducing inflammation as described in the present disclosure.

Alternatively, formulations and nutritional compositions belonging to the present invention may be considered to be nutraceuticals. As used herein, the term “nutraceutical” is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, soft-gelTM caplets, gel-caps and the like, not associated with food.

Extracts suitable for use in the present invention may be produced by extraction methods as are known and accepted in the art such as, but not limited to alcoholic extractions, aqueous extractions, and carbon dioxide extractions, for example. Other extraction methods will readily apparent to those of skill in the art.

It is herein understood that the activity of signaling molecules such as leukotrienes or chemotactic cytokines are the result of the activity of a mature protein or molecule. As such, the activity of said signaling molecules can be affected by modulating a number of non-mutually exclusive mechanisms. Such mechanisms include but are not limited to: transcription, translation, post-translation modification, dimerization, synthesis, secretion, degradation and receptor recognition and binding.

In is also herein understood that the activity of enzymes such as cyclooxygenase can be modulated by affecting a number of non-mutually exclusive mechanisms. Such mechanisms include but are not limited to: transcription, translation, post-translational modification, dimerization, substrate recognition and binding, catalytic activity and the binding of cofactors.

The present invention is directed towards a composition and method for reducing inflammation brought about by innate immune responses wherein said composition acts by inhibiting leukotrienes, chemotactic cytokines and cyclooxygenases, wherein the method comprises the oral administration of an effective amount of a composition comprising an extract of Butterbur, Bromelain and an extract of White Willow Bark.

Butterbur

Butterbur (Petasides hybridus) is a perennial shrub found throughout Europe, parts of North America and Asia. Butterbur has been clinically shown to be as effective as a common antihistamine, cetirizine, for treating seasonal allergic rhinitis (Schapowal A. Petasites Study Group. Randomised controlled trial of butterbur and cetirizine for treating seasonal allergic rhinitis. BMJ. 2002 Jan. 19; 324(7330):144-6). Asthma has also been effectively treated with Butterbur extract (Danesch UC. Petasites hybridus (Butterbur root) extract in the treatment of asthma—an open trial. Altern Med Rev. 2004 March; 9(1):54-62). Butterbur is thought to act in treating inflammation-related conditions such as allergies by inhibiting the biosynthesis of leukotrienes through one of its main active constituents, petasine (Monograph. Petasites hybridus. Altern Med Rev. 2001 April; 6(2):207-9).

Studies on the administration of extracts of Butterbur, which have been standardized to 8 mg of petasines, to 330 patients who suffer from intermittent allergic rhinitis (IAR) have shown the extracts of Butterbur are as effective in alleviation of IAR symptoms as the antihistamine drug, fexofenadine (Schapowal A, Study Group. Treating intermittent allergic rhinitis: a prospective, randomized, placebo and antihistamine-controlled study of butterbur extract Ze 339. Phytother Res. 2005 June; 19(6):530-7). The petasine-rich butterbur extract, Ze 339, exerts its antiallergic effects in part due to petasines inhibiting the biosynthesis of leukotriene, and related proinflammatory cytokines (Schapowal A. Butterbur Ze 339 for the treatment of intermittent allergic rhinitis. Dose-dependent efficacy in a prospective, randomized, double-blind, placebo-controlled study. Arch Otolaryngol Head Neck Surg. 2004 December; 130(12):1381-6).

It is herein understood by the inventors that Butterbur extract acts to reduce inflammation via inhibition of leukotriene synthesis. Furthermore, it is herein understood that administration of Butterbur extract as a dietary supplement will confer the benefits noted herein attributed to Butterbur.

In an embodiment of the present invention which is set forth in greater detail in the example below, the composition includes an extract of Butterbur to inhibit the production of leukotrienes. A serving of the composition comprises from about 0.050 g to about 0.500 g of Butterbur extract. The preferred dosage of an extract of Butterbur in the present invention comprises about 0.157 g per serving.

In certain embodiments a portion of the extract of Butterbur may be fine-milled. In particular, a fine-milled portion of Butterbur extract may be added beyond that of the preferred non-fine-milled dosage. Preferably, at least 0.003 g of Butterbur extract per serving is fine-milled. In a further embodiment the surface coating of acceptable oral dosage formats, as discussed below, may be imbued with a portion of Butterbur extract which is fine-milled. The inclusion of fine-milled Butterbur extract in the coating provides a rapid release of the Butterbur extract, specifically. Rapid release of the Butterbur extract immediately acts to inhibit the production of leukotrienes thereby quelling the pool of cytokines available to induce inflammation.

Bromelain

Bromelain is a glycoprotein enzyme with proteolytic activity derived from the stem of the pineapple plant (Ananas comosos). Bromelain has anti-inflammatory properties and has been successful at improving the symptoms of arthritis and knee pain (Walker A F, Bundy R, Hicks S M, Middleton R W. Bromelain reduces mild acute knee pain and improves well-being in a dose-dependent fashion in an open study of otherwise healthy adults. Phytomedicine. 2002 December; 9(8):681-6; Akhtar N M, Naseer R, Farooqi A Z, Aziz W, Nazir M. Oral enzyme combination versus diclofenac in the treatment of osteoarthritis of the knee—a double-blind prospective randomized study. Clin Rheumatol. 2004 October; 23(5):410-5). The efficacy of bromelain has been theorized to be due multiple activities unrelated to its proteolytic activity, including the ability to block various chemokine mediators of inflammation (Bromelain. Altern Med Rev. 1998 August; 3(4):302-5; Bromelain: biochemistry, pharmacology and medical use. Cell Mol Life Sci. 2001 August; 58(9):1234-45).

It is herein understood by the inventors that Bromelain acts to reduce inflammation by blocking the activity of chemokine-mediators of inflammation. Furthermore, it is herein understood that administration of Bromelain as a dietary supplement will confer the benefits noted herein attributed to Bromelain.

In an embodiment of the present invention which is set forth in greater detail in the example below, the composition comprises Bromelain to block the activity of chemokine-mediators of inflammation. A serving of the composition comprises from about 0.0005 g to about 0.005 g of Bromelain. The preferred dosage of Bromelain in the present invention comprises about 0.001 g per serving.

White Willow Bark

White Willow bark (Salix alba) is a source of salicin, a precursor of acetylsalicylic acid (aspirin) traditionally used to treat pain, fever and inflammation. White Willow bark extract has been shown to be as effective as a synthetic NSAID for relieving lower back pain (Chrubasik S, Kunzel 0, Model A, Conradt C, Black A. Treatment of low back pain with a herbal or synthetic anti-rheumatic: a randomized controlled study. Willow bark extract for low back pain. Rheumatology (Oxford). 2001 December; 40(12):1388-93). As a source of salicin, the anti-inflammatory mechanism of White Willow bark is understood to be mediated via the inhibition of cyclooxygenase (Vane J R. The fight against rheumatism: from willow bark to COX-1 sparing drugs. J Physiol Pharmacol. 2000 December; 51(4 Pt 1):573-86).

It is herein understood by the inventors that White Willow bark acts to reduce pain and inflammation by blocking the activity of cyclooxygenase enzymes, thereby inhibiting the production of mediators of inflammation. Furthermore, it is herein understood that administration of White Willow bark as a dietary supplement will confer the benefits noted herein attributed to White Willow bark.

In an embodiment of the present invention which is set forth in greater detail in the example below, the composition comprises White Willow bark extract to block the activity cyclooxygenase enzymes. A serving of the composition comprises from about 0.0005 g to about 0.005 g of White Willow bark extract. The preferred dosage of White Willow bark extract in the present invention comprises about 0.001 g per serving.

While, not wishing to be bound by theory, it is believed that the composition of the present invention will act to provide relief to individuals through consumption of said composition by affecting multiple mechanisms of innate immunity substantially simultaneously. Butterbur extract will act to inhibit the synthesis of leukotrienes. Bromelain will act to block the activity of chemokine mediators of inflammation. White Willow bark extract will act to inhibit the activity of cyclooxygenase enzymes. These non-mutually exclusive actions will jointly and substantially simultaneously act to reduce inflammation brought about by innate immune responses. It is herein understood by the inventors that the administration of the composition of the present invention to a mammal will reduce inflammation and pain and it is important that the multiple target mechanisms are affected at substantially the same time.

It is understood by the inventors that individuals whom suffer from inappropriate or excessive inflammation brought about by innate immune responses would benefit from a composition and method for aiding in alleviation of the symptoms of such a condition. The present invention provides a composition for the substantial simultaneous delivery of compounds to alleviate the symptoms of inflammation brought about by innate immune responses. By way of oral administration of the composition of the present invention, a method is provided for the alleviation of the symptoms of inflammation brought about by innate immune responses.

According to various embodiments of the present invention, the nutritional composition may be consumed in any form. For instance, the dosage form of the nutritional composition may be provided as, e.g., a powder beverage mix, a liquid beverage, a dietary gel, a ready-to-eat bar or drink product. Preferably, acceptable oral dosage formats are provided as: a capsule, a liquid capsule, a tablet. a caplet, a time-release capsule, a time-release liquid capsule, a time-release tablet, or as a time-release caplet. The preferred dosage form of the present invention is that of a caplet.

Embodiments of the present invention may employ particle-milling technology for enhanced utility and efficacy. U.S. patent application Ser. No. 11/709,526 entitled “Method For Increasing The Rate And Consistency Of Bioavailability Of Supplemental Dietary Ingredients” filed Feb. 21, 2007, which is herein fully incorporated by reference discloses the use of particle-milling for the purposes of increasing the rate of bioavailability following oral administration of components comprising supplemental dietary compositions. The increased bioavailability of a compound or ingredients is achieved via a reduction in particle size using a “fine-milling” technique. For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role. The components of the present invention may fine-milled in order to quicken the rate of dissolution.

Additionally, U.S. patent application Ser. No. 11/709,525 entitled “Method for a Supplemental Dietary Composition Having a Multi-Phase Dissolution Profile” filed Feb. 21, 2007, also herein fully incorporated by reference, discloses that components of the present invention may be used as portions of both non-milled and fine-milled, in order to provide a bi-phasic dissolution profile. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Further to such additional embodiments, the components of the present invention may be present in portions fine-milled to varying degrees thereby providing a multi-phasic dissolution profile as is disclosed in the preceding application reference.

The dosage form of the supplemental composition may be provided in accordance with customary processing techniques for herbal and nutritional compositions in any of the forms mentioned above. Additionally, the supplemental composition set forth in the example embodiment herein may contain any appropriate number and type of excipients, as is well known in the art.

Furthermore, by way of ingestion of the composition of the present invention, a method for reducing inflammation due to an innate immune response is provided.

The present nutritional composition or those similarly envisioned by one of skill in the art may be utilized in methods to reduce inflammation due to an innate immune response in an individual.

Although the following example illustrates the practice of the present invention in one of its various embodiments, the example should not be construed as limiting the scope of the invention. Other embodiments will be apparent to one skilled in the art from consideration of the specification of the following example.

Extensions and Alternatives

In the foregoing specification, the invention has been described with a specific embodiment thereof; however, it will be evident that various modifications and changes may be made thereto without departing from the broader spirit and scope of the invention.

### EXAMPLE

A nutritional composition is provided in one serving per day as two caplets per serving. In an embodiment of the present invention, each serving of the nutritional composition comprises:
about 0.155 g of extract of Butterbur root, about 0.003 g of fine-milled extract of Butterbur root, about 0.001 g of Bromelain and about 0.001 g of extract of White Willow bark.

The nutritional composition is administered to a mammal as required once daily to reduce the inflammation associated with inappropriate or excessive innate immune responses.

What is claimed:

1. A composition for reducing the inflammation brought about by innate immune responses in a mammal comprising;
from about 0.05 g to about 0.5 g of an extract of Butterbur;
from about 0.0001 g to about 0.01 g of Bromelain;
and from about 0.0001 g to about 0.01 g of an extract of White Willow Bark.

2. The composition of claim 1, wherein the amount of the extract of Butterbur is about 0.157 g; the amount of Bromelain is about 0.0010 g; and the amount of extract of White Willow Bark is about 0.0010 g.

3. The composition of claim 1, wherein the extract of Butterbur comprises petasines.

4. The composition of claim 1, wherein;
the extract of Butterbur, the Bromelain and the extract of White Willow Bark act to inhibit the innate immune response.

5. The composition of claim 1, wherein said composition is provided to a mammal in need thereof in an acceptable oral dosage format.

6. The composition of claim 5, wherein the acceptable oral dosage format is selected from the group consisting of tablets, caplets, capsules and soft-gel capsules.

7. The composition of claim 6, wherein the acceptable oral dosage format is provided in a form selected from the group consisting of unmodified release and time-release.

8. The composition of claim 6, wherein the oral dosage format comprises unmodified release in combination with a time-release format.

9. The composition of claim 7, where in the time-release formats are selected from the group of controlled-release, slow-release, delayed-release and quick-release.

10. The composition of claim 8, where in the time-release formats are selected from the group of controlled-release, slow-release, delayed-release and quick-release.

11. A method for reducing the inflammation brought about by innate immune responses in a mammal comprising:
administering to the mammal from about 0.05 g to about 0.5 g of an extract of Butterbur; from about 0.0001 g to about 0.01 g of Bromelain; and from about 0.0001 g to about 0.01 g of an extract of White Willow Bark.

12. The method of claim 11, wherein the extract of Butterbur comprises petasines.

13. The method of claim 11, wherein;
the extract of Butterbur, the Bromelain and the extract of White Willow Bark act to inhibit the innate immune response.

14. The method of claim 11, wherein said composition is provided to a mammal in need thereof in an acceptable oral dosage format.

15. The method of claim 14, wherein the acceptable oral dosage format is selected from the group consisting of tablets, caplets, capsules and soft-gel capsules.

16. The composition of claim 15, wherein the acceptable oral dosage format is provided in a form selected from the group consisting of unmodified release and time-release.

17. The composition of claim 15, wherein the oral dosage format consists of an unmodified release in combination with a time-release format.

18. The composition of claim 16, where in the time-release formats are selected from the group of controlled-release, slow-release, delayed-release and quick-release.

19. The composition of claim 17, where in the time-release formats are selected from the group of controlled-release, slow-release, delayed-release and quick-release.
